(19) United States
(12) Patent Application Publication
(10) Pub. No.: US 2024/0079142 A1
(43) Pub. Date: Mar. 7, 2024

(54) A SYSTEM AND METHOD TO PREDICT HEALTH OUTCOMES AND OPTIMIZE HEALTH INTERVENTIONS

(71) Applicant: Manifold Inc., Oakland, CA (US)

(72) Inventors: Sourav Dey, Palo Alto, CA (US); Joshua Hayes, San Rafael, CA (US)

(21) Appl. No.: 18/269,542

(22) PCT Filed: Dec. 27, 2021
(86) PCT No.: PCT/US2021/065232

§ 371 Date: Jun. 23, 2023

Related U.S. Application Data
(60) Provisional application No. 63/169,022, filed on Mar. 31, 2021.
(60) Provisional application No. 63/131,276, filed on Dec. 28, 2020.

(51) Int. Cl. G16H 50/20; G16H 50/30; G16H 50/50; G16H 50/70

(52) U.S. Cl. CPC G16H 50/20; G16H 50/30; G16H 50/50; G16H 50/70

(57) ABSTRACT

A method to predict health outcomes is provided. The method includes receiving data for a first population comprising data for individuals who have undergone a therapy and data for individuals in a control group who have not undergone the therapy, and identifying biomarker values for the first population. The method includes creating data for a second population based on the biomarker values, and determining a first distribution of health outcomes for individuals in the second population. The method includes determining a second distribution of health outcomes for individuals in the second population, and evaluating a quality of the therapy based on a difference between the first distribution of health outcomes and the second distribution of health outcomes. A system and a non-transitory computer readable medium storing instructions to cause the system to perform the above method are also provided.

### CROSS-REFERENCE TO RELATED APPLICATIONS

The present disclosure is related and claims priority under 35 U.S.C. § 119(e) to U.S. Provisional Pat. Appln. No. 63/131,276 entitled ENGINE FOR PREDICTING THE OUTCOME FOR A HEALTHCARE THERAPY, to Dey, filed on Dec. 28, 2020, and to U.S. Provisional Pat. Appln. No. 63/169,022 entitled A SYSTEM AND METHOD TO OPTIMIZE INTERVENTIONS FOR HEALTH, to Dey, et al. filed on Mar. 31, 2021, the contents of which are hereby incorporated by reference in their entirety, for all purposes.

### BACKGROUND

### Field

The present disclosure is related to optimizing interventions to improve health outcomes based on real world evidence. More specifically, embodiments as disclosed herein provide a system and method to direct interventions to patients who are most likely to benefit based on causal relationships between the intervention and the outcome.

### Related Art

Machine learning can be used to predict health outcomes, but current machine learning methods rely on correlations in data. Correlation, however, does not equal causation. For example, β-amyloid plaques are correlated with Alzheimer's disease, and machine learning can predict Alzheimer's disease in patients from MRI or CT scans of the brain. However, β-amyloid plaques may not cause Alzheimer's disease, and certain drugs that target β-amyloid have failed to show improvements in slowing the progression of Alzheimer's disease. Machine learning models may be able to predict with high accuracy a health outcome such as Alzheimer's disease, but predictive models are not sufficient to determine appropriate intervention (e.g., drug treatment).

Randomized controlled trials (RCTs) (e.g., clinical trials) have traditionally been used to evaluate causal relationships. For example, in an RCT, one group is randomly assigned to receive an intervention, while a control group does not receive the intervention. The efficacy and safety of the intervention is measured by comparing the outcomes of the groups. Confidence in the causal relationship between two variables is increased because the researcher has taken steps to control for all possible confounding relationships. But RCTs are time-consuming, expensive, and sometimes impossible. The embodiments disclosed herein provide a more efficient and effective evaluation of interventions using real-world data.

### BRIEF DESCRIPTION OF THE DRAWINGS

FIG. 1 illustrates a system architecture for predicting and improving the outcome of a healthcare therapy, according to some embodiments.

FIGS. 2A and 2B illustrate servers, devices, processors, memories, and circuits to implement the system architecture of FIG. 1, according to some embodiments.

FIG. 3 illustrates a cost distribution comparison between two different populations in a healthcare therapy, according to some embodiments.

FIGS. 4A-4B illustrate diagrams to implement a causal discovery for a healthcare therapy, according to some embodiments.

FIGS. 5A-5C illustrate different statistical tools in a causal inference engine, according to some embodiments.

FIG. 6 illustrates a block chart for using causal graphs and causal modeling to obtain average treatment effects and conditional average treatment effects including a counterfactual model, according to some embodiments.

FIG. 7 illustrates a reward distribution for a contextual bandits (CB) tool in a causal inference engine, according to some embodiments.

FIG. 8 is a flowchart illustrating steps in a method for selecting and performing an intervention with a causal inference engine, according to some embodiments.

FIG. 9 is a flowchart illustrating steps in a method to evaluate a healthcare therapy based on real world data, according to some embodiments.

FIG. 10 is a flowchart illustrating steps in a method for selecting a group of patients to target an intervention, according to some embodiments.

FIG. 11 is a flowchart illustrating steps in a method to estimate cause effect modifications in a causal inference engine, according to some embodiments.

FIG. 12 illustrates a system to implement the architecture and perform the various methods illustrated in FIGS. 1, 2A-2B, and in the flowcharts in FIGS. 8-11, according to some embodiments.

In the figures, elements with like or similar reference numerals have like or similar descriptions, unless stated explicitly, otherwise.

### SUMMARY

In a first embodiment, a computer-implemented method includes receiving data for a first population including data for multiple individuals who have undergone a therapy and data for multiple individuals in a control group who have not undergone the therapy; identifying biomarker values for the first population; creating data for a second population based on the biomarker values; determining a first distribution of health outcomes for individuals in the second population who are simulated to undergo the therapy; determining a second distribution of health outcomes for individuals in the second population who are similar to individuals in the control group of the first population; and evaluating a quality of the therapy based on a difference between the first distribution of health outcomes and the second distribution of health outcomes.

In a second embodiment, a system includes one or more processors, and a memory storing multiple instructions, wherein the one or more processors execute the instructions to cause the system to perform operations. The operations include to receive data for a first population including data for multiple individuals who have undergone a therapy and data for multiple individuals in a control group who have not undergone the therapy; identify biomarker values for the first population; create data for a second population based on the biomarker values; determine a first distribution of health outcomes for individuals in the second population who are simulated to undergo the therapy; determine a second distribution of health outcomes for individuals in the second population who are similar to individuals in the control group of the first population; and evaluate a quality of the therapy based on a difference between the first distribution of health outcomes and the second distribution of health outcomes.

In a third embodiment, a computer-implemented method includes receiving observational data associated with a medical treatment; estimating a treatment effect from the observational data; selecting one or more individuals for an intervention based on the treatment effect; and estimating the outcome of the intervention.

### DETAILED DESCRIPTION

In the following detailed description, numerous specific details are set forth to provide a full understanding of the present disclosure. It will be apparent, however, to one ordinarily skilled in the art, that embodiments of the present disclosure may be practiced without some of these specific details. In other instances, well-known structures and techniques have not been shown in detail so as not to obscure the disclosure.

In the field of digital healthcare, drug manufacturers, clinicians, and payers (e.g., health insurance companies, government agencies, and the like) interact to provide therapeutic procedures for subjects that are effective for resolving a healthcare problem and also economically viable. One of the challenges is to identify a cohort of subjects that may benefit most from a given therapeutic procedure. In some instances, specific sub-populations from a large population may have substantive benefits from a given therapeutic, making it economically viable by appropriate pricing. Identifying these sub-populations a-priori is difficult in the context of a randomized controlled trial (RCT). In addition, RCTs tend to select smaller groups of populations and test for a limited amount of time (e.g., weeks or months). It is desirable to arrive to accurate results that may be projected over longer periods of time (years, decades, or even a lifetime of a subject). Embodiments as disclosed herein provide an engine that identifies these sub-populations when they exist, and accurately evaluate the cost-benefit of tailoring a therapeutic procedure to this selected cohort of the population.

To achieve this, an engine as disclosed herein incorporates a causal inference approach to randomized control trial (RCT) data to estimate the healthcare outcomes of the therapy on a larger dataset. Healthcare outcomes may include a number of insurance claims, hospital or doctor office visits, or medical costs. In some embodiments, the original RCT is enlarged by selecting randomized ‘look alike’ subjects from real world data based on biomarker distributions inferred from the original RCT. With the larger data set, a more accurate estimate of healthcare outcomes may be determined for a selected sub-population (‘target group’) of patients or subjects. Moreover, the causal inference approach in engines as disclosed herein provides answers to counterfactual queries, such as an accurate prediction of what would be the response of a given subject or a cohort of subjects to a specific action or item in the therapy, or if a specific action or item in the therapy is modified in a certain manner. In some embodiments, an engine as disclosed herein may provide insight to evaluate a therapy based on the health and cost impacts of the adherence of a patient to the therapy. In some embodiments, an engine as disclosed herein may indicate the costs associated with hypertension based on a cardiometabolic treatment.

In addition to using causal inference to estimate and identify causal effects in observed data patterns, other machine learning techniques may be used such as supervised learning, to learn from examples; unsupervised learning, to find hidden structure in data sets; and reinforcement learning to act and iterate on an identified hypothesis based on prediction success. Bayesian methods may be used to estimate distributions of biomarker changes. In addition, embodiments as disclosed herein make use of computer networking techniques to run Monte Carlo (MC) simulations over large population datasets.

In addition to medicines and medical procedures, other aspects of general healthcare management for patients may include habits such as hygiene procedures, eating habits, and exercising habits. Accordingly, systems as disclosed herein may aggregate data associated with the above habits that may be collected by mobile devices used by patients as they go about their normal lives (e.g., wearable devices measuring steps or exercise performed by a person, mobile devices that capture shopping baskets at a grocery store, or collect information about the venues and places visited by a patient). Systems as collected herein collect and aggregate the above information and provide recommendations (e.g., an intervention with the patient) with the goal of improving a health outcome for the patients.

An intervention includes an action upon a variable that can be controlled, modified, or adjusted to affect a health outcome. For example, an intervention may include a pharmaceutical drug, medical procedure, or medical device. An intervention may include an adjustment to a drug dosage or dosing schedule, timing of a medical procedure, or setting on a medical device. An intervention may also include coaching or other efforts to modify the patient's behavior, such as messages delivered to a patient, a reminder in an application, or one or more phone calls from the healthcare provider.

Some embodiments include a human-mediated step in the loop, such as recommendation to a physician. In some embodiments, a fully automated system, such as fully automated messaging, may be implemented with reinforcement learning approaches (e.g., “contextual bandits,” and the like).

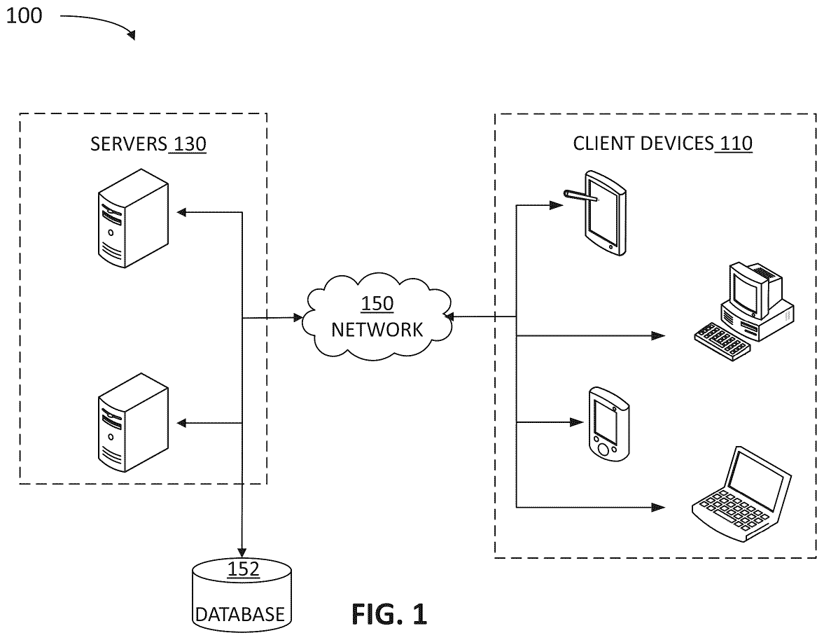

FIG. 1 illustrates a system architecture 100 for predicting and improving the outcome of a healthcare therapy, according to some embodiments. Architecture 100 includes servers 130 communicatively coupled with client devices 110 and at least one database 152 over a network 150. One of the many servers 130 is configured to host a memory including instructions which, when executed by a processor, cause the server 130 to perform at least some of the steps in methods as disclosed herein. In some embodiments, the processor is configured to control a graphical user interface (GUI) in an application for the user of one of client devices 110 accessing a healthcare intervention optimization server or a causal inference engine. The application may include a healthcare provider application (accessed by a healthcare provider), a patient application (accessed by a patient), or a patient messaging application (to send messages, reminders, and alerts to a patient, according to a healthcare intervention strategy).

The healthcare intervention optimization server may be configured to train a machine learning model for solving a specific healthcare therapeutic question. Accordingly, the processor may include a dashboard tool, configured to display components and graphic results to the user via the GUI. For purposes of load balancing, multiple servers 130 can host memories including instructions to one or more processors, and multiple servers 130 can host a history log and databases including multiple training archives used for the healthcare intervention optimization server. Moreover, in some embodiments, multiple users of client devices 110 may access the same healthcare intervention optimization server to run one or more machine learning models. In some embodiments, a single user with a single client device 110 may train multiple machine learning models running in parallel in one or more servers 130. Accordingly, client devices 110 may communicate with each other via network 150 and through access to one or more servers 130 and resources located therein.

Servers 130 may include any device having an appropriate processor, memory, and communications capability for hosting the healthcare intervention optimization server or the engine, including multiple tools associated with it. The healthcare intervention optimization server may be accessible by various clients 110 over network 150. Client devices 110 can be, for example, desktop computers, mobile computers, tablet computers (e.g., including e-book readers), mobile devices (e.g., a smartphone or PDA), or any other device having appropriate processor, memory, and communications capabilities for accessing the healthcare intervention optimization server on one or more of servers 130. Network 150 can include, for example, any one or more of a local area tool (LAN), a wide area tool (WAN), the Internet, and the like. Further, network 150 can include, but is not limited to, any one or more of the following tool topologies, including a bus network, a star network, a ring network, a mesh network, a star-bus network, tree or hierarchical network, and the like.

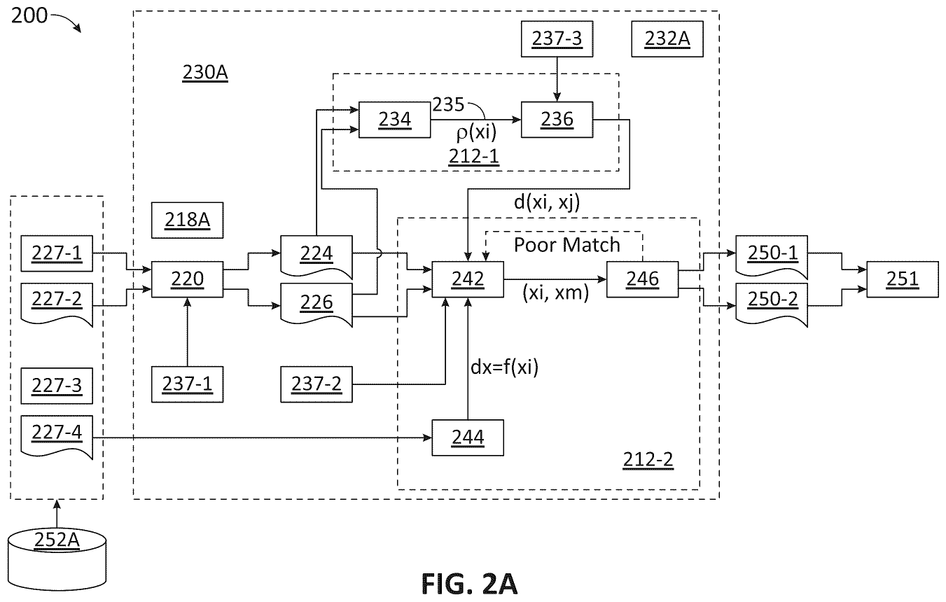

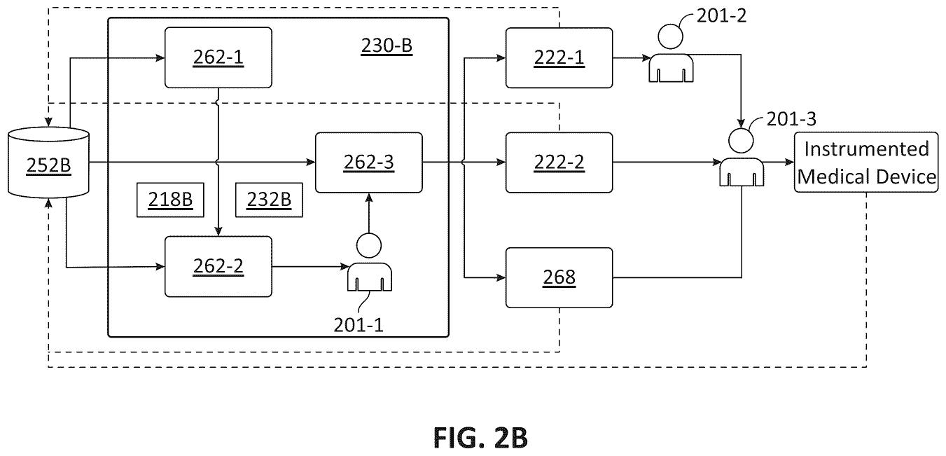

FIGS. 2A and 2B illustrate servers 230A and 230B (hereinafter, collectively referred to as “servers 230”), devices, modules 212-1 and 212-2 (hereinafter, collectively referred to as “modules 212”), memories 232A and 232B (hereinafter, collectively referred to as “memories 232”), databases 252A and 252B (hereinafter, collectively referred to as “databases 252”) and circuits to implement a system architecture as disclosed herein (cf. system architecture 100), according to some embodiments. Some of the blocks may include hardware and software interacting via the processors and memories in one or more computers distributed over a network (cf. system architecture 100). Accordingly, the various circuits may be communicatively coupled with wired signals or wireless signals processed by communications modules 218A and 218B (hereinafter, collectively referred to as “communications modules 218”). Communications modules 218 are configured to interface with a network to send and receive information to servers 230, such as data, requests, responses, and commands to other devices on the network. Communications modules 218 can be, for example, modems or Ethernet cards, and may include radio hardware and software to produce and process signals in the form of electromagnetic radiation (e.g., radiofrequency, or RF), such as Wi-Fi, BlueTooth, NFC, and the like.

FIG. 2A illustrates a block diagram of a system 200 configured to perform at least some of the methods disclosed herein.

A treatment set splitter 220 separates treatment member data and control member data from a first population of subjects according to targeting criteria 227-1 applied to member data 227-2, based on hyper-parameters 237-1 (e.g., to target and split sample data). Member data 227-2 may include data retrieved from a healthcare provider database, a government database, or any other database including healthcare data from a first population of subjects (e.g., database 252A).

A distance metric definition module 212-1 includes a propensity score fit tool 234 that evaluates a propensity score 235 of being in the treatment group for any member. A distance metric creator tool 236 determines a distance metric by comparing propensity score 235 between individuals, given hyperparameters 237-3 as input (e.g., exact match requirements, propensity calipers, and the like). Distance metric definition module 212-1 may use a propensity caliper and set a threshold for the distance metric.

An MC matching module 212-2 uses distance metric data provided by distance metric definition module 212-1 and an MC delta biomarker sampler 244 using RCT data 227-3 and prior therapeutic data 227-4 to a feed matcher module 242. Matcher module 242 finds and refines matches in matcher module 242 with treatment member data 224 and control member data 226, over an enlarged dataset, using hyper-parameters 237-2 (e.g., number of trials, number of matches, and the like). Hyper-parameters 237-1 and 237-2 will be collectively referred to, hereinafter, as “hyper-parameters 237.” A matched diagnostics 246 is sorted into treatment member data healthcare outcomes and healthier looking member healthcare data outcomes. The distribution of healthcare outcomes and claims 250-1 (treated patients) and 250-2 (untreated patients), hereinafter, collectively referred to as “healthcare outcomes and claims 250,” for the two groups can be compared. For example, a distribution 251 of healthcare outcomes and claims 250 may indicate an economic impact of the therapy as applied to the selected subject population.

In some embodiments, intervention optimization server 230B includes a predictive model engine 262-1, a causal inference engine 262-2, and a randomized experiment engine 262-3 (hereinafter, collectively referred to as “engines 262”). Engines 262 collect observational data from database 252B. In some embodiments, a designer 201-1 may manually intervene to verify the operations of engines 262. For example, in some embodiments, designer 201-1 may manually include a hypothesis, such as a causal relationship of variables specified in a directed acyclic graph, or identify a sub-population to test a heterogeneous treatment effect, in a given healthcare optimization task.

A provider application 222-1 may be run by a healthcare provider 201-2 to control or monitor an RCT developed by intervention optimization server 230B. A patient 201-3 may participate in the RCT via a patient application 222-2. Provider application 222-1 and patient application 222-2 (hereinafter, collectively referred to as “applications 222”) may be installed in client devices (e.g., a desktop computer, a laptop, a smartphone, and the like) communicatively coupled to intervention optimization server 230B and to one another via a network. For example, patient 201-3 may participate in the RCT via a patient messaging engine 268, that provides communication between patient 201-3 and intervention optimization engine 230B.

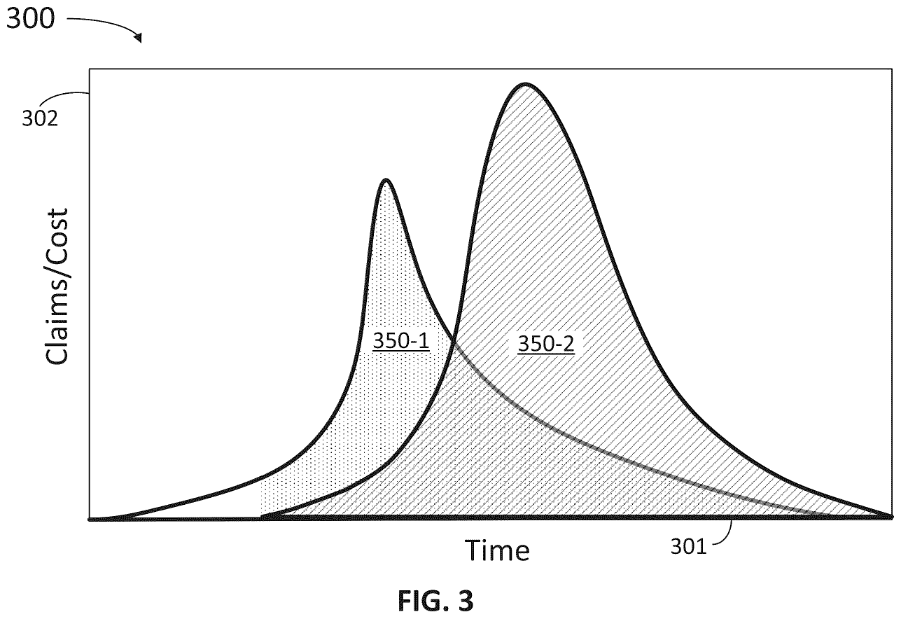

FIG. 3 illustrates a cost distribution 300 for comparison between two different sub-populations 350-1 and 350-2 (hereinafter, collectively referred to as “sub-populations 350”) in a healthcare therapy, according to some embodiments. The abscissae 301 (X-axis) indicates time (e.g., months, or years), and the ordinates 302 (Y-axis) indicate a number of claims (e.g., health insurance cost) accrued for treatment of a certain illness or health condition. Sub-population 350-1 may include patients that have undergone a therapy whose performance is being evaluated, and sub-population 350-2 may include a control population of patients that did not go through the therapy. By looking at the area under cost distribution 300, it is seen that overall, it is more cost-efficient to follow the therapy than not. In some embodiments, such a cost differential may be included as a factor driving interventions and other actions in the healthcare therapy.

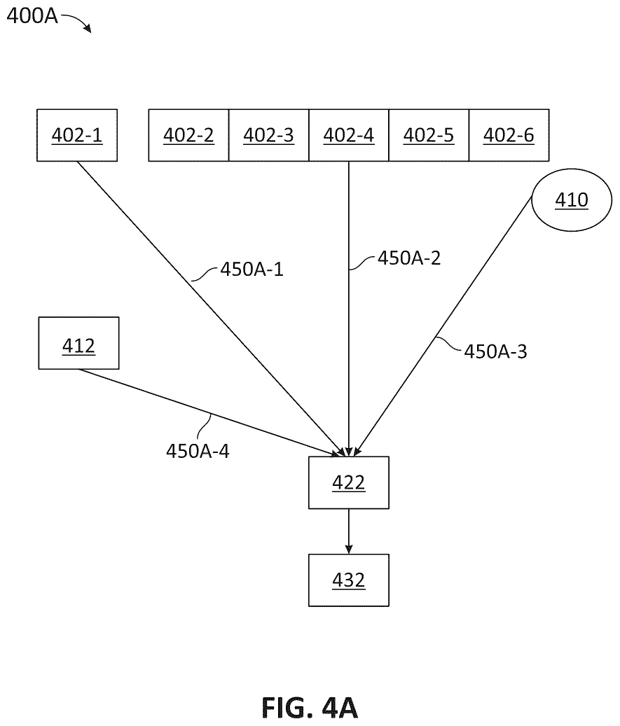

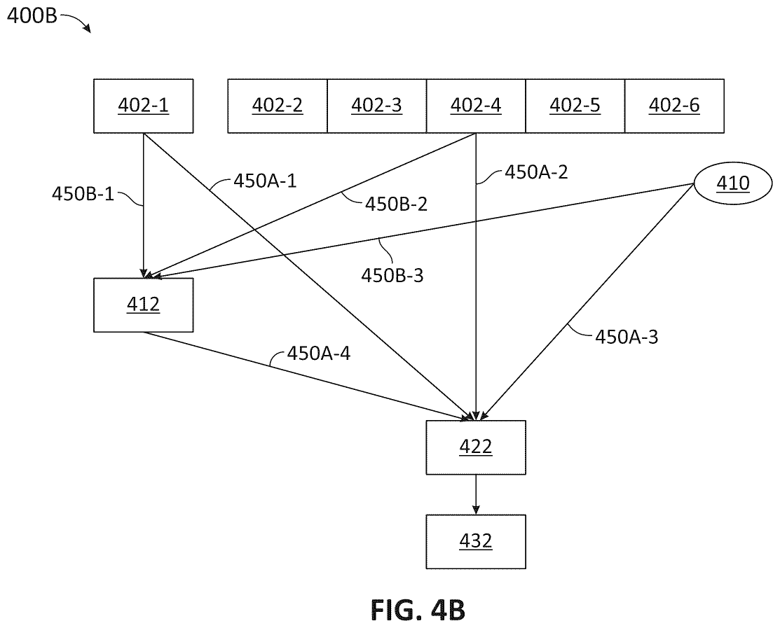

FIGS. 4A-4B illustrate directed acyclic graphs (DAGs) 400A and 400B (hereinafter, collectively referred to as “DAGs 400”) to implement causal discovery analysis in design of an RCT for a healthcare therapy, according to some embodiments. DAGs 400 simplify RCT design because they provide a precise estimation of cause factors 402-1 (e.g., prior usage of a prescribed medication), 402-2 (e.g., a reason or motivation for use of the prescribed medication), 402-3 (e.g., a motivator), 402-4 (e.g., disease state), 402-5 (e.g., diagnostic factor), and 402-6 (e.g., gender, age, and other demographic factors), hereinafter, collectively referred to as “cause factors 402.” DAGs 400 also include actions 412 that may be included in the RCT (e.g., notifications—e-mails, SMS, push—sent to patients for medicine intake, reminders, and the like). More specifically, DAGs 400 offer a simple manner to identify unobserved confounders 410.

DAGs 400 link cause factors 402, actions 412, and unobserved confounders 410 to a result 422 (e.g., medication usage by the patient), and an outcome 432 (e.g., compliance) via direct links 450A-1 (e.g., medication usage prior to notification prompting medication usage after notification). 450A-2 (e.g., other prompts), 450A-3 (e.g., an unobserved confounder affecting medication usage), 450A-4 (e.g., a notification sent to the patient prompting medication usage) and 450B-1 (e.g., medication usage prompting a notification to the patient), 450B-2 (e.g., other prompts), and 450B-3 (e.g., unobserved confounder 410 affecting the notifications sent to the patient), hereinafter, collectively referred to as “links 450.” The strength (thickness), and color of links 450 indicate the predicted weight of cause factor 402 or action 412, to produce result 422 and outcome 432.

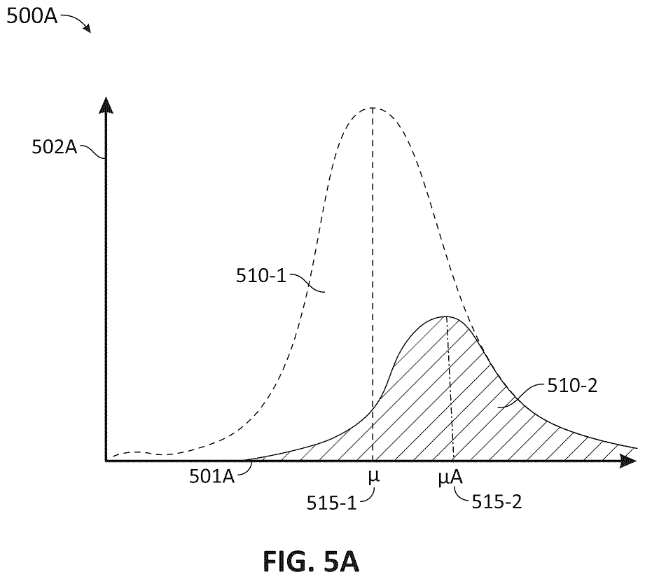

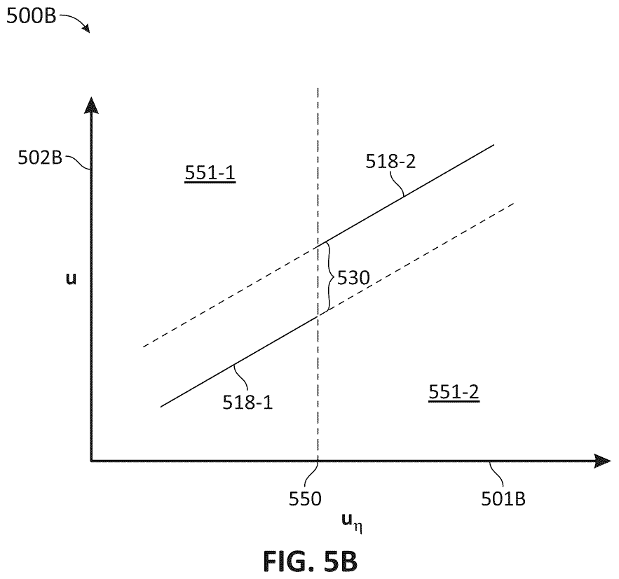

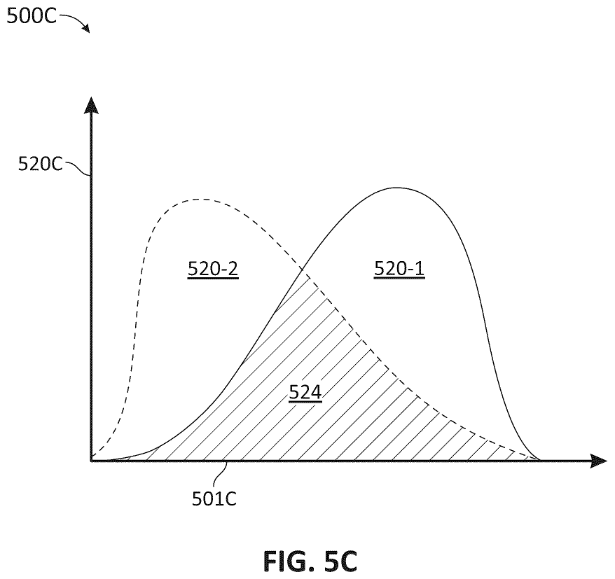

FIGS. 5A-5C illustrate charts 500A, 500B, and 500C (hereinafter, collectively referred to as “charts 500”) from statistical tools in a causal inference engine, according to some embodiments. Charts 500 can be the result of observational studies to perform RCTs. In some embodiments, charts 500 use existing historical data to generate useful hypothesis for RCT. Charts 500 are designed to understand and identify confounding factors that may obscure causal relationships (e.g., confounding factors 410).

Observational studies use historical data on a given patient population to perform statistical analysis on measurable outcomes (OT, for treated patients, and OU, for untreated patients). Some of the statistical parameters that a causal inference engine may determine include an average treatment effect (ATE), using the following expression:

ATE=E[O_{T}]−E[O_{U}] (1)

Where E[x] is the statistical expected value calculation of variable x. ATE measures a causal signal and may depend on counterfactual information (e.g., confounding factors that may obscure or negate a seemingly direct causal relationship). The presence of confounding factors are a challenge for solving Eq. 1. Additionally, observational data for any given patient can only be either ‘treated’ or ‘untreated’, as the same patient can only belong to either one of these categories. Accordingly, a true solution for Eq. 1 is challenging. Another observational study may include a simple difference of outcomes (SDO), defined as:

SDO=E[O_{T}|X=1]−E[O_{U}|X=0] (2)

Wherein X=1 means that a patient was treated, and X=0 means that the patient was untreated. SDO measures association (e.g., correlation) between the measured outcome and the treatment, and is directly measurable. In general, Eqs. (1) and (2) provide different values, as ATE (cf. Eq. 1) is a counterfactual estimate and cannot be directly observed because we cannot see the counterfactual world. SDO (cf. Eq. 2) is merely measuring the difference in the two treated subsets, but does not take into account differences that are not due to the causal effect, e.g. it is not controlling for confounders. In a truly ‘random,” unbiased RCT, ATE is equal to SDO. This is not always the case, as there may be confounding factors that introduce a selection bias in the observed samples.

Chart 500A illustrates a heterogeneous treatment effect (HTE), which is obtained by determining a conditional average treatment effect (CATE), according to some embodiments. The abscissae (X-axis) 501A indicates a value of a random variable (e.g., the probability that a given patient will take medication on schedule, or will satisfy a certain measured outcome in treatment). The ordinates 502A (Y-axis) indicate a number of counts, or frequency, for elements within a main population (curve 510-1) or from a sub-population (curve 510-2). The statistical factors, e.g., mean value 515-1 (main population) and mean value 515-2 (curve 510-2), hereinafter collectively referred to as “statistical factors 515,” may be quite different for the main population and for the sub-population.

CATE can be calculated based on the following expression:

CATE_{A}=E[O_{T}|A]−E[O_{U}|A] (3)

Wherein E[OT|A] is the expected outcome value for patients in the treated sub-population, A, and E[OU|A] is the expected outcome value for patients in the un-treated sub-population A. Chart 500A clearly illustrates that sub-population A may respond more strongly (or weakly) to a given treatment. Some factors that may be determinant in sub-population A may include demographic factors such as age and gender, psychological factors such as engagement, or physical factors such as treatment type, and the like. When every measure of CATE (Eq. 3) is equal to ATE (Eq. 1), then there is no HTE in the population sample.

Table I below shows an exemplary result of CATE calculations for a number of (hypothetical) patients.

TABLE 1

| | | Feature | Feature | Feature | | |
|---|---|---|---|---|---|---|
| Patient | Date | 1 | 2 | 3 | CATEA | CATEB |
| A | Sep. 1, 2020 | 0.11 | 77 | XX | 0.31 | 0.1 |
| A | Aug. 31, 202 | 0.12 | 76 | YY | 0.02 | 0.44 |
| B | Sep. 1, 2020 | 0.85 | 12 | ZZ | 0.01 | 0.03 |
| B | Aug. 31, 2020 | 0.79 | 11 | ZZ | 0.15 | 0.22 |
| C | Sep. 1, 2020 | 0.55 | 44 | XX | 0.21 | .01 |

Chart 500B illustrates a discontinuity observed at an intervention point. Typically, an intervention is made on a selected value 550 along a continuous variable 501B (e.g., historical usage). Selected value 550 separates a regression 518-1 for a control group 551-1 from a regression 518-2 for a treatment group 551-2 (hereinafter, collectively referred to as “patient groups 551” and “regressions 518”). The ordinates 502B (Y-axis) indicate any value associated with regressions 518. A gap value 530 in chart 500B may be attributed to the average treatment effect (ATE). Accordingly, gap value 530 may provide a measure for the ATE, and thus, chart 500B is a good indicator on whether a certain causal hypothesis may be true or may be associated with a confounding factor.

Chart 500C illustrates an observational method including matching treated population area 520-1 and untreated population area 520-2 (hereinafter, collectively referred to as “patient population areas 520”), according to some embodiments. The abscissae 501C indicates a selected random variable (e.g., probability of treatment), and the ordinates 502C indicate a frequency count for patients within the selected random variable range. Chart 500C may be used to identify confounder factors. Users are binned into categories (e.g., age, gender, previous medication usage, and the like), and a balance between treated/untreated patients may be observable. For example, matched area 524 is used to estimate the ATE (cf. Eq. 1). Matched area 524 enables a causal effect estimation for random variable 501C (e.g., if the overlap is too small, establishing a causality is more difficult, due to the lack of similarity between the two populations 520-1 and 520-2), random variable 501C may be discarded as a causal factor.

A propensity score is a generalization of chart 500C including more random variables in the analysis. Propensity scores enable a more robust match between treated and untreated populations 520. In some embodiments, propensity scores enable a match across a range of factors at once, without need for splitting the sample populations into subsets, and thus allowing a larger overlapped matching set. This enables a more accurate measurement of causal effects (e.g., ATE, cf. Eq. 1) by using a larger data set for matching.

In addition to the above observational tools, a causal inference engine as disclosed herein may perform other operations, such as “difference in difference,” and “instrumental variables.”

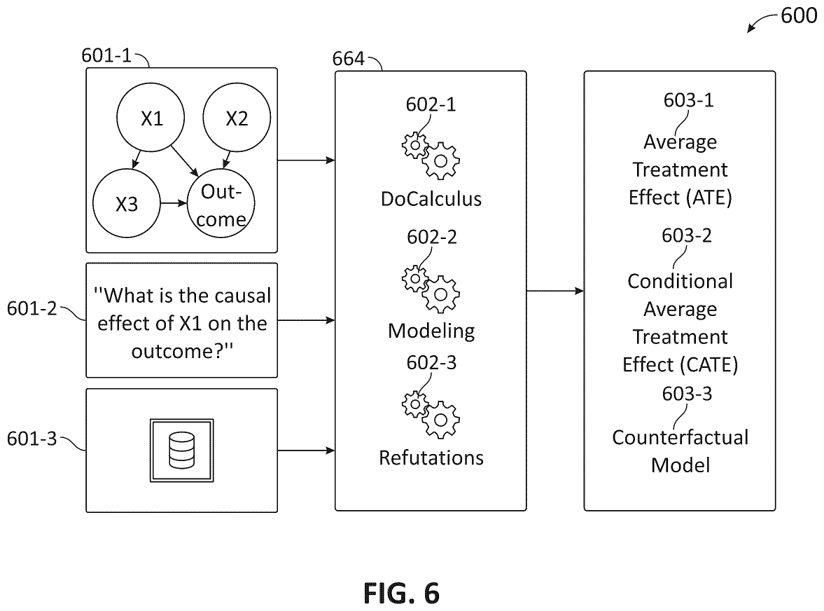

FIG. 6 illustrates a block chart 600 for using DAGs 601-1, causal modeling 601-2, and observational data 601-3 (hereinafter, collectively referred to as “inputs 601”) to obtain average treatment effects 603-1 and conditional average treatment effects 603-2, including a counterfactual model 603-3 (hereinafter, collectively referred to as “outputs 603”), according to some embodiments.

A causal inference engine 664 includes a calculating tool 602-1, a modeling tool 602-2, and a refutations tool 602-3 (hereinafter, collectively referred to as “tools 602”) that operate on inputs 601, to generate outputs 603.

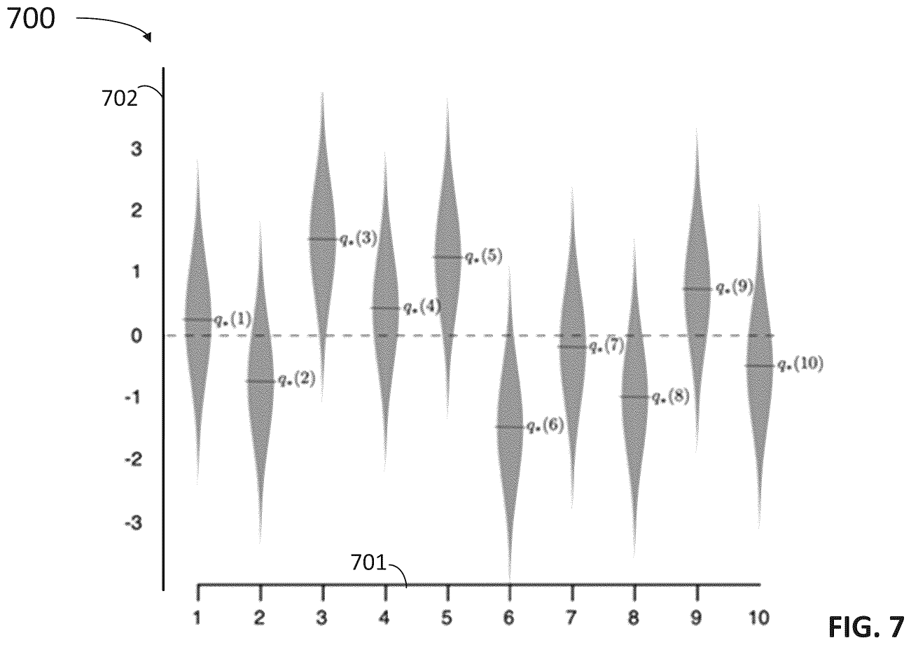

FIG. 7 illustrates a reward distribution 700 for a contextual bandits (CB) tool in a causal inference engine, according to some embodiments. Reward distributions 702 are plotted around multiple actions (e.g., interventions) 701 that may be carried. The CB tool identifies the combination of actions that renders a higher likelihood of a positive reward, or the highest positive reward attainable.

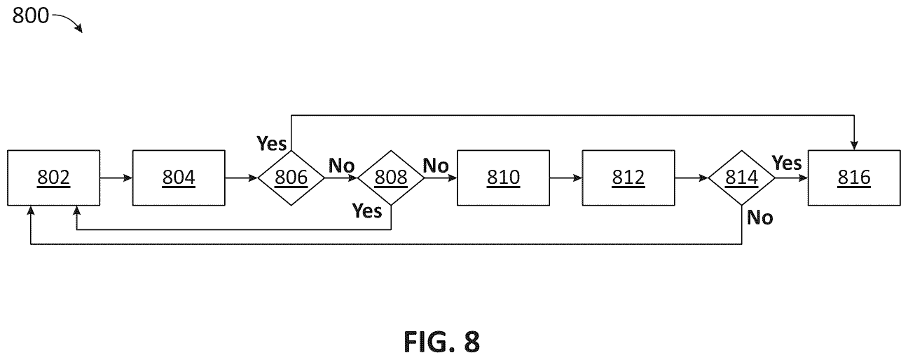

FIG. 8 is a flow chart illustrating steps in a method 800 for selecting and performing an intervention with a causal inference engine, according to some embodiments. In some embodiments, method 800 may be performed at least partially by one or more processors executing instructions stored in a memory or database (e.g., memories 232, databases 152 and 252). In some embodiments, method 800 includes steps performed at least partially by an intervention optimization server, including a predictive model, a causal inference engine, and a randomized experiment engine, as disclosed herein (e.g., intervention optimization server 230B, predictive model engine 262-1, causal inference engine 264, and RCT engine 266). In some embodiments, methods consistent with the present disclosure may include one or more steps in method 800, performed in a different sequence, simultaneously, quasi-simultaneously, or overlapping in time.

Step 802 includes developing hypotheses on appropriate interventions based on domain knowledge.

Step 804 includes testing the hypothesis with causal inference on observational data. Accordingly, in some embodiments, step 804 validates the hypothesis, and identifies patients that have high, or the highest CATE/HTE to treatment. In some embodiments, step 804 includes measuring outcome on adherence of the intervention in patients.

Step 806 includes evaluating whether the treatment effect of the hypothetical intervention is significant.

When step 806 finds that the treatment effect is not significant, 808 includes determining whether step 806 is answerable with observational data.

When step 808 finds that step 806 may not be answerable with observational data, step 810 includes testing the intervention with RCT.

Step 812 includes measuring the adherence outcome of the intervention in patients, with causal inference on the RCT data. The outcome may be an adherence forecast. In some embodiments, the outcome may be heterogeneous across patients.

Step 814 includes assessing whether the adherence outcome produces a significant enough change in treatment effect to validate the intervention.

When step 814 finds that the treatment effect is significant, step 816 includes deploying the intervention to a targeted population.

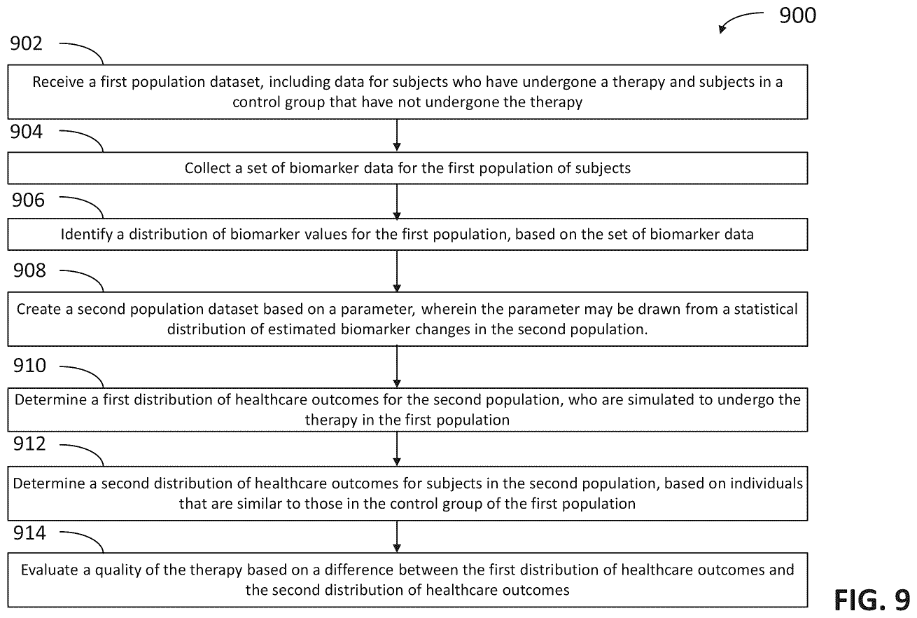

FIG. 9 is a flowchart illustrating steps in a method 900 to evaluate a healthcare therapy based on real world data, according to some embodiments. In some embodiments, method 900 may be performed at least partially by one or more processors executing instructions stored in a memory or database (e.g., memories 232, databases 152 and 252). In some embodiments, at least one or more of the steps in method 900 may be performed by an MC matching module, including an MC delta biomarker sampler and a matcher module (e.g., MC matching module 212-2, MC delta biomarker 244, and matcher module 242). In some embodiments, at least one or more of the steps in method 900 may be performed by a distance metric definition module including a propensity score fit tool, a distance metric creator tool, and a propensity caliper (e.g., distance metric definition module 212-1, propensity score fit tool 234, and distance metric creator tool 236). In some embodiments, method 900 includes steps performed at least partially by an intervention optimization server, including a predictive model, a causal inference engine, and a randomized experiment engine, as disclosed herein (e.g., intervention optimization server 230B, predictive model engine 262-1, causal inference engine 264, and RCT engine 266). In some embodiments, methods consistent with the present disclosure may include one or more steps in method 900, performed in a different sequence, simultaneously, quasi-simultaneously, or overlapping in time.

Step 902 includes receiving a first population dataset, including data for subjects who have undergone a therapy and subjects in a control group that have not undergone the therapy. In some embodiments, step 902 includes receiving demographic data such as gender or age. In some embodiments, step 902 includes receiving medical condition data. In some embodiments, step 902 includes selecting a first population of subjects including multiple subjects to undergo a therapy and multiple subjects in a control group that do not undergo the therapy.

Step 904 includes collecting a set of biomarker data from the first population of subjects. In some embodiments, step 904 includes collecting a Cholesterol value or a blood sugar value.

Step 906 includes identifying a distribution of biomarker values for the first population, based on the set of biomarker data.

Step 908 includes creating a second population dataset based on a parameter. In some embodiments, the second population is a larger population dataset based on a statistical parameter from the distribution of biomarker values. In some embodiments, step 908 includes evaluating a random variable for a biomarker value conditioned by the statistical parameter and comparing a difference between the random variable and the set of biomarker data with a distance metric derived by a propensity caliper.

In some embodiments, step 908 includes drawing the parameter from a statistical distribution of estimated biomarker changes in the second population. In some embodiments, step 908 includes comparing the distance metric of demographic attributes between members of the first population and a second population.

Step 910 includes determining a first distribution of healthcare outcomes for the second population, who are simulated to undergo the therapy in the first population. In some embodiments, step 910 includes determining a first distribution of health outcomes or claims for subjects in the larger (e.g., second) population dataset, based on subjects that look like subjects that underwent the therapy in the first population of subjects. In some embodiments, step 910 includes predicting the set of biomarker data after a selected period of time for subjects in the second population based on a supervised learning model. In some embodiments, step 910 includes predicting the set of biomarker data after a selected period of time for subjects in the second population based on a socio-economic parameter associated with the first population of subjects. In some embodiments, step 910 includes predicting the set of biomarker data after a selected period of time for subjects in the second population based on a therapy feature. In some embodiments, step 910 includes predicting the set of biomarker data after a selected period of time based on a genomic data from the first population of subjects. In some embodiments, step 910 includes classifying the set of biomarker data based on a random forest algorithm. In some embodiments, step 910 includes classifying the set of biomarker data based on a deep learning algorithm.

Step 912 includes determining a second distribution of healthcare outcomes for subjects in the second population, based on individuals that are similar to those in the control group of the first population. In some embodiments, step 912 includes determining a second distribution of health outcomes or claims for subjects in the larger (e.g., the second) population dataset, based on subjects that look like subjects in the control group.

Step 914 includes evaluating a quality of the therapy based on a difference between the first distribution of healthcare outcomes or values and the second distribution of healthcare outcomes or values. In some embodiments, step 914 further includes adjusting a demographic filter for selecting the first population of subjects to undergo the therapy based on the quality of the therapy. In some embodiments, step 914 further includes selecting at least one biomarker from a group consisting of a Cholesterol value and a blood sugar value.

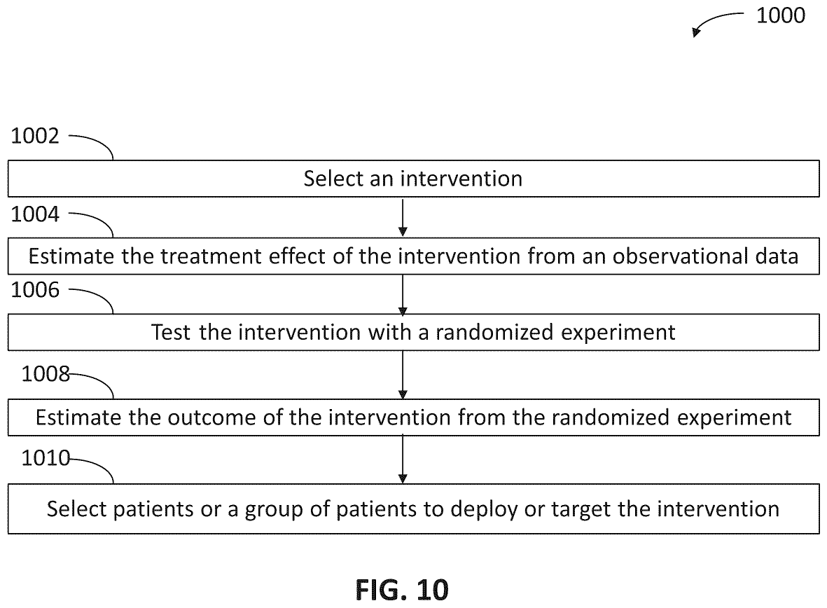

FIG. 10 is a flowchart illustrating steps in a method 1000 for selecting a group of patients to target an intervention, according to some embodiments. In some embodiments, method 1000 may be performed at least partially by one or more processors executing instructions stored in a memory or database (e.g., memories 232, databases 152 and 252). In some embodiments, method 1000 includes steps performed at least partially by an intervention optimization server, including a predictive model, a causal inference engine, and a randomized experiment engine, as disclosed herein (e.g., intervention optimization server 230B, predictive model engine 262-1, causal inference engine 264, and RCT engine 266). In some embodiments, methods consistent with the present disclosure may include one or more steps in method 1000, performed in a different sequence, simultaneously, quasi-simultaneously, or overlapping in time.

Step 1002 includes selecting an intervention.

Step 1004 includes estimating the treatment effect of the intervention from an observational data.

Step 1006 includes testing the intervention with a randomized experiment.

Step 1008 includes estimating the outcome of the intervention from the randomized experiment.

Step 1010 includes selecting patients or a group of patients to deploy or target the intervention.

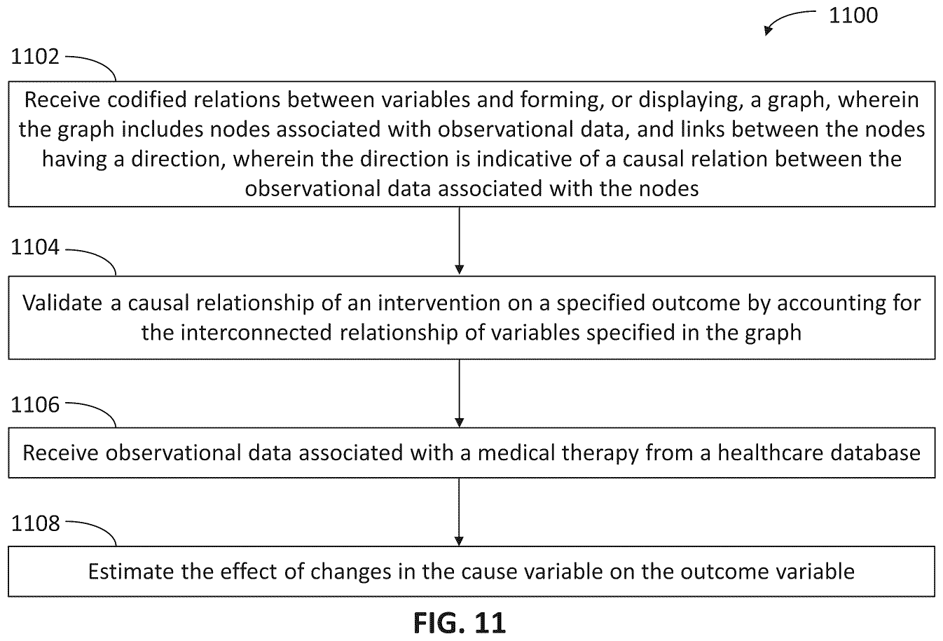

FIG. 11 is a flowchart illustrating steps in a method 1100 to estimate causal effect modifications in a causal inference engine, according to some embodiments. In some embodiments, method 1100 may be performed at least partially by one or more processors executing instructions stored in a memory or database (e.g., memories 232, databases 152 and 252). In some embodiments, method 1100 includes steps performed at least partially by an intervention optimization server, including a predictive model, a causal inference engine, and a randomized experiment engine, as disclosed herein (e.g., intervention optimization server 230B, predictive model engine 262-1, causal inference engine 264, and RCT engine 266). In some embodiments, methods consistent with the present disclosure may include one or more steps in method 1100, performed in a different sequence, simultaneously, quasi-simultaneously, or overlapping in time.

Step 1102 includes receiving codified relations between variables and forming, or displaying, a graph, wherein the graph includes nodes associated with observational data, and links between the nodes having a direction, wherein the direction is indicative of a causal relation between the observational data associated with the nodes.

Step 1104 includes validating a causal relationship of an intervention on a specified outcome by accounting for the interconnected relationship of variables specified in the graph.

Step 1106 includes receiving observational data associated with a medical therapy from a database.

Step 1108 includes estimating the effect of changes in the cause variable on the outcome variable.

### Hardware Overview

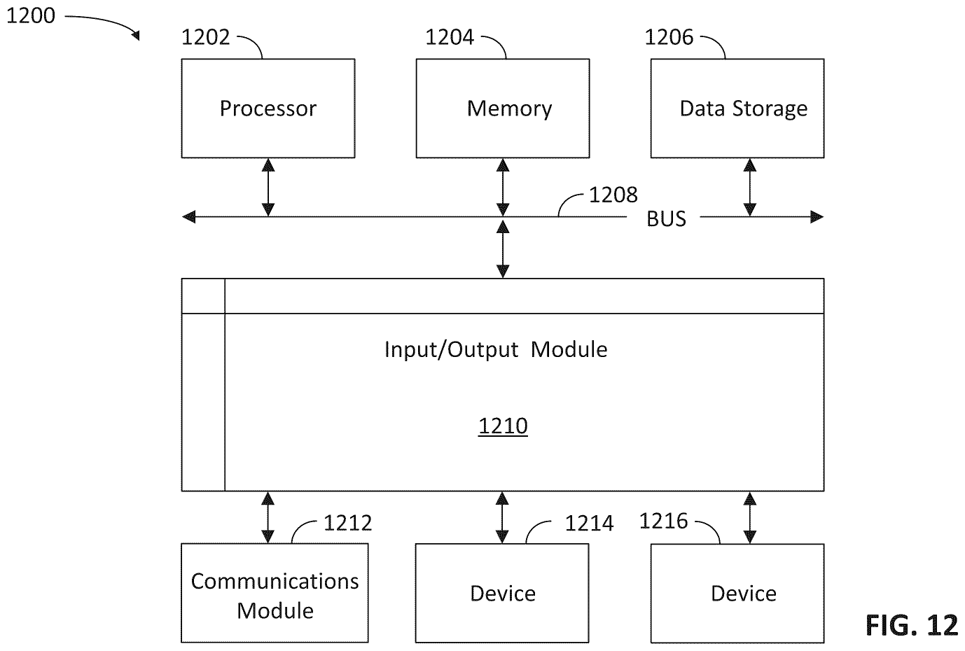

FIG. 12 illustrates a system 1200 to implement the architecture and perform the various methods illustrated in FIGS. 1, 2A-2B, and in the flowcharts in FIGS. 8-11, according to some embodiments. In certain aspects, the computer system 1200 may be implemented using hardware or a combination of software and hardware, either in a dedicated server, or integrated into another entity, or distributed across multiple entities.

Computer system 1200 (e.g., system 200) includes a bus 1208 or other communication mechanism for communicating information, and a processor 1202 (e.g., distance metric definition module 212-1, MC matching module 212-2) coupled with bus 1208 for processing information. By way of example, the computer system 1200 may be implemented with one or more processors 1202. Processor 1202 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1200 can include, in addition to hardware, code that creates an execution environment for the computer program in question, e.g., code that constitutes processor firmware, a protocol stack, a database management system, an operating system, or a combination of one or more of them stored in an included memory 1204 (e.g., databases 152 and 252), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled to bus 1208 for storing information and instructions to be executed by processor 1202. The processor 1202 and the memory 1204 can be supplemented by, or incorporated in, special purpose logic circuitry.

The instructions may be stored in the memory 1204 and implemented in one or more computer program products, e.g., one or more modules of computer program instructions encoded on a computer-readable medium for execution by, or to control the operation of, the computer system 1200, and according to any method well-known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 1204 may also be used for storing temporary variable or other intermediate information during execution of instructions to be executed by processor 1202.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 1200 further includes a data storage device 1206 such as a magnetic disk or optical disk, coupled to bus 1208 for storing information and instructions. Computer system 1200 may be coupled via input/output module 1210 to various devices. Input/output module 1210 can be any input/output module. Exemplary input/output modules 1210 include data ports such as USB ports. The input/output module 1210 is configured to connect to a communications module 1212. Exemplary communications modules 1212 (e.g., communications modules 218) include networking interface cards, such as Ethernet cards and modems. In certain aspects, input/output module 1210 is configured to connect to a plurality of devices, such as an input device 1214 (e.g., a keyboard, a mouse, a pointer, a touchscreen display, a microphone, a webcam, and the like) and/or an output device 1216 (e.g., a display, a touchscreen display, a speaker, and the like). Exemplary input devices 1214 include a keyboard and a pointing device, e.g., a mouse or a trackball, by which a user can provide input to the computer system 1200. Other kinds of input devices 1214 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 1216 include display devices, such as an LCD (liquid crystal display) monitor, for displaying information to the user.

According to one aspect of the present disclosure, the client and server can be implemented using a computer system 1200 in response to processor 1202 executing one or more sequences of one or more instructions contained in memory 1204. Such instructions may be read into memory 1204 from another machine-readable medium, such as data storage device 1206. Execution of the sequences of instructions contained in main memory 1204 causes processor 1202 to perform the process steps described herein. One or more processors in a multi-processing arrangement may also be employed to execute the sequences of instructions contained in memory 1204. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back-end component, e.g., a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. The communication network (e.g., network 150) can include, for example, any one or more of a LAN, a WAN, the Internet, and the like. Further, the communication network can include, but is not limited to, for example, any one or more of the following tool topologies, including a bus network, a star network, a ring network, a mesh network, a star-bus network, tree or hierarchical network, or the like. The communications modules can be, for example, modems or Ethernet cards.

Computer system 1200 can include clients and servers. A client and server are generally remote from each other and typically interact through a communication network. The relationship of client and server arises by virtue of computer programs running on the respective computers and having a client-server relationship to each other. Computer system 1200 can be, for example, and without limitation, a desktop computer, laptop computer, or tablet computer. Computer system 1200 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 1202 for execution. Such a medium may take many forms, including, but not limited to, non-volatile media, volatile media, and transmission media. Non-volatile media include, for example, optical or magnetic disks, such as data storage device 1206. Volatile media include dynamic memory, such as memory 1204. Transmission media include coaxial cables, copper wire, and fiber optics, including the wires forming bus 1208. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

In one aspect, a method may be an operation, an instruction, or a function and vice versa. In one aspect, a clause may be amended to include some or all of the words (e.g., instructions, operations, functions, or components) recited in either one or more clauses, one or more words, one or more sentences, one or more phrases, one or more paragraphs, and/or one or more clauses.

To illustrate the interchangeability of hardware and software, items such as the various illustrative blocks, modules, components, methods, operations, instructions, and algorithms have been described generally in terms of their functionality. Whether such functionality is implemented as hardware, software, or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B. or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience only and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The subject technology is illustrated, for example, according to various aspects described below. Various examples of aspects of the subject technology are described as numbered claims (claim 1, 2, etc.) for convenience. These are provided as examples, and do not limit the subject technology. The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

### RECITATION OF EMBODIMENTS

Embodiments as disclosed herein may include any one of the following.

Embodiment I: A computer-implemented method that includes receiving data for a first population including data for multiple individuals who have undergone a therapy and data for multiple individuals in a control group who have not undergone the therapy, identifying biomarker values for the first population, creating data for a second population based on the biomarker values, determining a first distribution of health outcomes for individuals in the second population who are simulated to undergo the therapy, determining a second distribution of health outcomes for individuals in the second population who are similar to individuals in the control group of the first population, and evaluating a quality of the therapy based on a difference between the first distribution of health outcomes and the second distribution of health outcomes.

Embodiment II: A system that includes one or more processors, and a memory storing multiple instructions, wherein the one or more processors execute the instructions to cause the system to perform operations. The operations include receiving data for a first population including data for multiple individuals who have undergone a therapy and data for multiple individuals in a control group who have not undergone the therapy, identifying biomarker values for the first population, creating data for a second population based on the biomarker values, determining a first distribution of health outcomes for individuals in the second population who are simulated to undergo the therapy, determining a second distribution of health outcomes for individuals in the second population who are similar to individuals in the control group of the first population, and evaluating a quality of the therapy based on a difference between the first distribution of health outcomes and the second distribution of health outcomes.

Embodiment III: A computer-implemented method that includes receiving observational data associated with a medical treatment, estimating a treatment effect from the observational data, selecting one or more individuals for an intervention based on the treatment effect, and estimating the outcome of the intervention.

In addition to embodiments I, II and III above, further embodiments consistent with the present disclosure may include any permutation and number of the above embodiments and features with the following elements.

Element 1, wherein determining a first distribution of health outcomes includes predicting biomarker values for individuals in the second population. Element 2, wherein determining a first distribution of health outcomes includes predicting biomarker values for individuals in the second population based on a socio-economic parameter associated with the first population. Element 3, wherein determining a first distribution of health outcomes includes predicting biomarker values for individuals in the second population based on a therapy feature. Element 4, wherein determining a first distribution of health clauses includes predicting the set of biomarker data after a selected period of time based on a genomic data from the first population of subjects. Element 5, wherein determining a first distribution of health outcomes includes classifying the biomarker values based on a random forest. Element 6, wherein determining a first distribution of health outcomes includes classifying the biomarker values based on deep learning. Element 7, further including selecting individuals to undergo the therapy based on the evaluated quality of the therapy. Element 8, wherein creating data for a second population includes drawing a parameter from a statistical distribution of estimated biomarker values for individuals in the second population. Element 9, wherein creating data for a second population includes comparing a propensity score between individuals in the first population and a second population and selecting individuals from the second population with a propensity score below a propensity caliper.

Element 10, wherein to determine a first distribution of health outcomes the one or more processors execute instructions to predict biomarker values for individuals in the second population. Element 11, wherein to determine a first distribution of health outcomes the one or more processors execute instructions to predict biomarker values for individuals in the second population based on a socio-economic parameter associated with the first population. Element 12, wherein to determine a first distribution of health outcomes the one or more processors execute instructions to predict biomarker values after a selected period of time for individuals in the second population based on a therapy feature.

Element 13, wherein the treatment effect includes ATE or CATE. Element 14, wherein selecting one or more individuals for an intervention based on the treatment effect includes selecting one or more individuals based on the CATE value. Element 15, wherein the intervention includes a message sent to the one or more individuals. Element 16, wherein estimating the outcome of the intervention includes forecasting adherence to the treatment. Element 17, wherein estimating the outcome of the intervention includes estimating the outcome of the intervention based on a randomized experiment.

1. A computer-implemented method, comprising:
receiving data for a first population comprising data for multiple individuals who have undergone a therapy and data for multiple individuals in a control group who have not undergone the therapy;
identifying biomarker values for the first population;
creating data for a second population based on the biomarker values;
determining a first distribution of health outcomes for individuals in the second population who are simulated to undergo the therapy;
determining a second distribution of health outcomes for individuals in the second population who are similar to individuals in the control group of the first population; and
evaluating a quality of the therapy based on a difference between the first distribution of health outcomes and the second distribution of health outcomes.

2. The computer-implemented method of claim 1, wherein determining a first distribution of health outcomes comprises predicting biomarker values for individuals in the second population.

3. The computer-implemented method of claim 1, wherein determining a first distribution of health outcomes comprises predicting biomarker values for individuals in the second population based on a socio-economic parameter associated with the first population.

4. The computer-implemented method of claim 1, wherein determining a first distribution of health outcomes comprises predicting biomarker values for individuals in the second population based on a therapy feature.

5. The computer-implemented method of claim 1, wherein determining a first distribution of health clauses comprises predicting a set of biomarker data after a selected period of time based on a genomic data from the first population of subjects.

6. The computer-implemented method of claim 1, wherein determining a first distribution of health outcomes comprises classifying the biomarker values based on a random forest.

7. The computer-implemented method of claim 1, wherein determining a first distribution of health outcomes comprises classifying the biomarker values based on deep learning.

8. The computer-implemented method of claim 1, further comprising selecting individuals to undergo the therapy based on the quality of the therapy.

9. The computer-implemented method of claim 1, wherein creating data for a second population comprises drawing a parameter from a statistical distribution of estimated biomarker values for individuals in the second population.

10. The computer-implemented method of claim 1, wherein creating data for a second population comprises comparing a propensity score between individuals in the first population and a second population and selecting individuals from the second population with a propensity score below a propensity caliper.

11. A system, comprising:
one or more processors; and
a memory storing multiple instructions, wherein the one or more processors execute the instructions to cause the system to perform operations, comprising:
receiving data for a first population comprising data for multiple individuals who have undergone a therapy and data for multiple individuals in a control group who have not undergone the therapy;
identifying biomarker values for the first population;
creating data for a second population based on the biomarker values;
determining a first distribution of health outcomes for individuals in the second population who are simulated to undergo the therapy;
determining a second distribution of health outcomes for individuals in the second population who are similar to individuals in the control group of the first population; and
evaluating a quality of the therapy based on a difference between the first distribution of health outcomes and the second distribution of health outcomes.

12. The system of claim 11, wherein to determine a first distribution of health outcomes the one or more processors execute instructions to predict biomarker values for individuals in the second population.

13. The system of claim 11, wherein to determine a first distribution of health outcomes the one or more processors execute instructions to predict biomarker values for individuals in the second population based on a socio-economic parameter associated with the first population.

14. The system of claim 11, wherein to determine a first distribution of health outcomes the one or more processors execute instructions to predict biomarker values after a selected period of time for individuals in the second population based on a therapy feature.

15. A computer-implemented method, comprising:
receiving observational data associated with a medical treatment;
estimating a treatment effect from the observational data;
selecting one or more individuals for an intervention based on the treatment effect; and
estimating an outcome of the intervention.

16. The computer-implemented method of claim 15, wherein the treatment effect comprises ATE or CATE.

17. The computer-implemented method of claim 15, wherein selecting one or more individuals for an intervention based on the treatment effect comprises selecting one or more individuals based on a CATE value.

18. The computer-implemented method of claim 15, wherein the intervention comprises a message sent to the one or more individuals.

19. The computer-implemented method of claim 15, wherein estimating the outcome of the intervention comprises forecasting adherence to the medical treatment.

20. The computer-implemented method of claim 15, wherein estimating the outcome of the intervention comprises estimating the outcome of the intervention based on a randomized experiment.
